(19) United States
(12) Patent Application Publication
(10) Pub. No.: US 2024/0216143 A1
(43) Pub. Date: Jul. 4, 2024

(54) Modular Glenoid Component for Use in a Universal Shoulder Prosthesis System

(71) Applicant: Raphael S.F. Longobardi, LLC, Old Tappan, NJ (US)

(72) Inventor: Raphael S.F. Longobardi, Old Tappan, NJ (US)

(21) Appl. No.: 18/288,856

(22) PCT Filed: Apr. 29, 2022
(86) PCT No.: PCT/US2022/026940

§ 371 Date: Oct. 30, 2023

Related U.S. Application Data
(60) Provisional application No. 63/181,597, filed on Apr. 29, 2021.

(51) Int. Cl. A61F 2/40; A61F 2/30

(52) U.S. Cl. CPC A61F 2/4081; A61F 2002/30331; A61F 2002/30604

(57) ABSTRACT

A modular glenoid component with an angled concave top surface for use with or as part of a modular shoulder prosthesis system that provides flexibility in shoulder replacements and ability to switch between a traditional anatomic Total Shoulder Replacement (ta-TSR) to a reverse Total Shoulder Replacement (r-TSR). The module glenoid component includes a base having the concave top surface angled from an anterior side to a posterior side such that the anterior side is higher than the posterior side. Optionally, the modular glenoid component includes a plug and/or a peripheral flange both of which when present extend in a direction away from a bottom surface of the base.

### I. FIELD OF THE INVENTION

The invention relates to a modular glenoid component with an angled concave top surface for use in (or as part of) a modular shoulder prosthesis system that provides for flexibility in shoulder replacements and allows for a more efficient switch for a patient between a traditional anatomic Total Shoulder Replacement (ta-TSR) to a reverse Total Shoulder Replacement (r-TSR). In at least one embodiment, the modular glenoid component is for use in ta-TSR.

### II. BACKGROUND OF THE INVENTION

TSRs have evolved over the last 70 years, with the greatest degree of its evolution occurring within the past 20 years. The understanding of the complexity of the shoulder has resulted in the ability to better treat the multiple conditions that afflict the shoulder. Glenohumeral arthritis ranges from simple to complex due to etiology and deformity. Post traumatic glenohumeral arthritis, along with the deformity of both the glenoid and humeral head present challenges for the shoulder arthroplasty surgeon. Similarly, the problem of rotator cuff deficiency and rotator cuff arthropathy has resulted in the development of treatment and prosthetic designs specific to address the loss of motion in the shoulder.

Currently, there are two types of TSR—traditional anatomic total shoulder replacement (ta-TSR) and reverse total shoulder replacement (r-TSR). Ta-TSR utilizes resurfacing of the humeral head and glenoid in the setting of an intact and functioning rotator cuff. Glenohumeral arthritis has been treated with ta-TSR, the current gold standard being the resurfacing of the humeral head with a stemmed or metaphyseal component along with a replacement of the humeral head articular portion with a Cobalt-Chromium (Co—Cr) implant. Modularity of the humeral components allows for appropriate sizing of the head in diameter and thickness to match the resected articular surface of the patient.

Currently, all commercial ta-TSR, and r-TSR require removal of all components, especially the glenoid components, when revising. In the specific case of revision from ta-TSR to r-TSR, a universal system allows for continued use of the glenoid and humeral baseplates, with removal and substitution of the ta-TSR articulating surfaces [glenoid and humeral head] with those of the r-TSR articulating surfaces [glenosphere and humeral cup]. A problem arises when a patient has anterior instability leading to potential humeral head sublocation anteriorly. This condition typically requires greater retroversion of the components, which requires changes in the version of the humeral head osteotomy, or further bone removal from the glenoid to provide a greater angle for the glenoid component to prevent anterior instability.

### III. SUMMARY OF THE INVENTION

A modular system allows the surgeon to achieve either exchanges, humeral or glenoid component, without an extravagant amount of equipment to be used, or more complex operative procedures to be performed. A truly versatile and modular system allows for a baseplate to accept either a traditional anatomic glenoid component or a reverse total shoulder glenosphere, without compromising long term security and function.

In at least one embodiment, the modular component is for the glenoid side in a ta-TSR and includes a base having an angled concave top surface and a plug extends from a surface opposite of the concave surface where the plug is configured to be inserted into the glenoid baseplate. The concave surface is configured to engage a humeral head, which in at least one embodiment includes at least a partially spherical surface. In a further embodiment, the angle results in a lower wall height on the posterior side as compared to a higher wall height on the anterior side of the modular glenoid component. In a further embodiment to the previous two embodiments, the modular glenoid component includes a pair of protrusions extending from opposing sides of the base, the protrusions configured to align with the notches when the modular glenoid component is attached to the base. In a further embodiment to the embodiments of this paragraph, the modular glenoid component includes a pair of protrusions extending down from opposing sides of the baseplate, the protrusions configured to engage with an interference fit the notches when the modular glenoid component is attached to the baseplate. In a further embodiment to the above embodiments, the modular glenoid component includes a flange extending down from the outer circumferential edge such that the flange fits over and/or around the baseplate. In such an embodiment with the protrusions, the pair of protrusions are present on the interior peripheral sides of the flange. In a further embodiment to any of the previous embodiments, the plug of the modular glenoid component includes a Morse taper. Further to the previous embodiments, the modular glenoid component includes Co—Cr. In a further embodiment to any of the previous embodiments, a universal should prosthesis system including the modular component.

### IV. BRIEF DESCRIPTION OF THE DRAWINGS

Any cross-hatching present in the figures is not intended to identify or limit the type of material present for the element shown in cross-section. In figures that include multiple elements shown in cross-section, the cross-hatching will be different directions for the different elements.

FIGS. 1A-1C illustrate a baseplate. FIG. 1A illustrates a top view. FIG. 1B illustrates a side view with phantom lines illustrating the internal construction. FIG. 1C illustrates an alternative cross-section of the baseplate for an alternative attachment with a glenosphere modular component from a view from superiorly of the glenosphere where the cross-section is taken along a diameter.

FIGS. 1D and 1E illustrate side views of alternative baseplates with phantom lines illustrating the internal construction according to at least two embodiments of the invention. FIG. 1F illustrates a side view with phantom lines illustrating the internal construction according to at least one embodiment of the invention.

FIGS. 1G-1J illustrate an alternative baseplate. FIGS. 1G and 1H illustrate perspective views from the top and bottom, respectively. FIG. 1I illustrates a side view. FIG. 1J illustrates a cross-section taken through a diameter through the center of the notches.

FIGS. 2A-2D illustrate a glenoid component depicting top, perspective, bottom, and cross-section views, respectively. FIG. 2D is a cross-section of a modular glenoid component engaged with a baseplate.

FIG. 2E illustrates an alternative embodiment to FIG. 2B.

FIGS. 2F-2H illustrate an alternative modular glenoid component. FIG. 2G is a cross-section taken at 2G-2G in FIG. 2F, which illustrates a top view of the modular glenoid component. FIG. 2H illustrates a side view of the modular glenoid component.

### V. DETAILED DESCRIPTION OF THE DRAWINGS

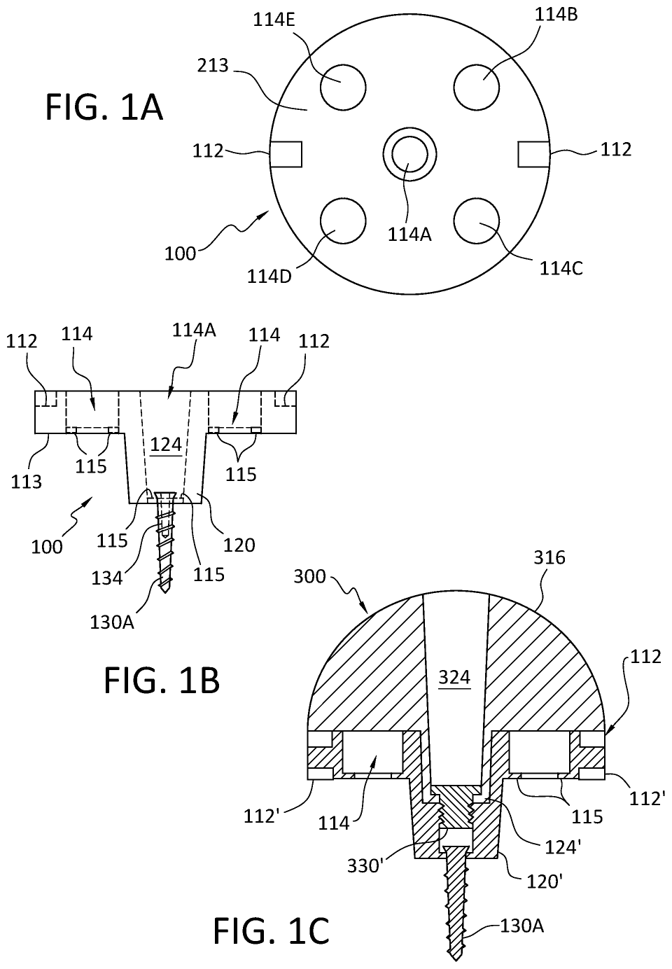

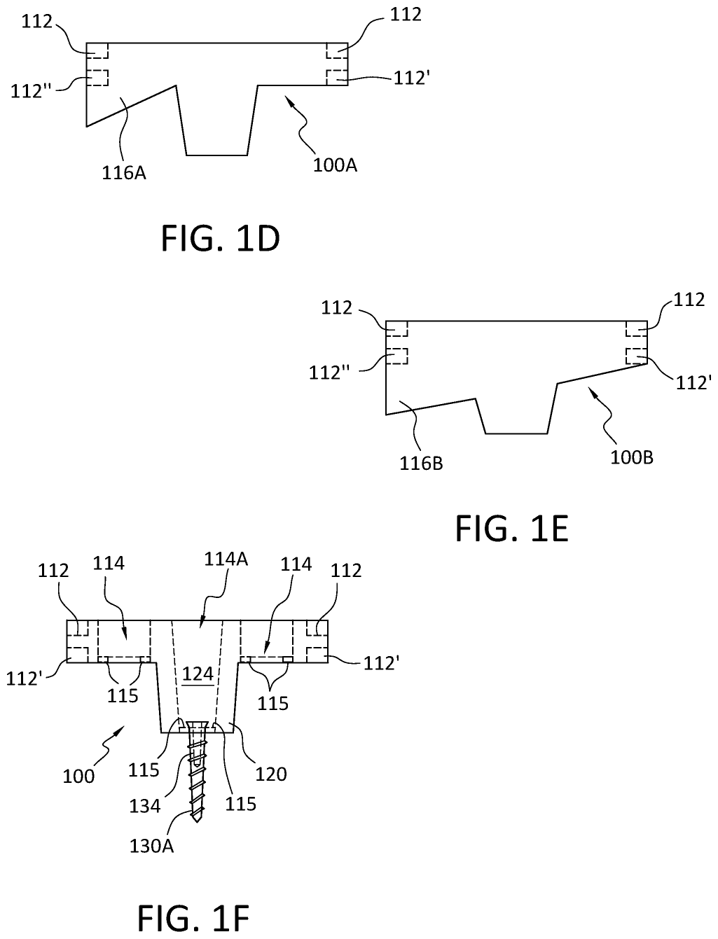

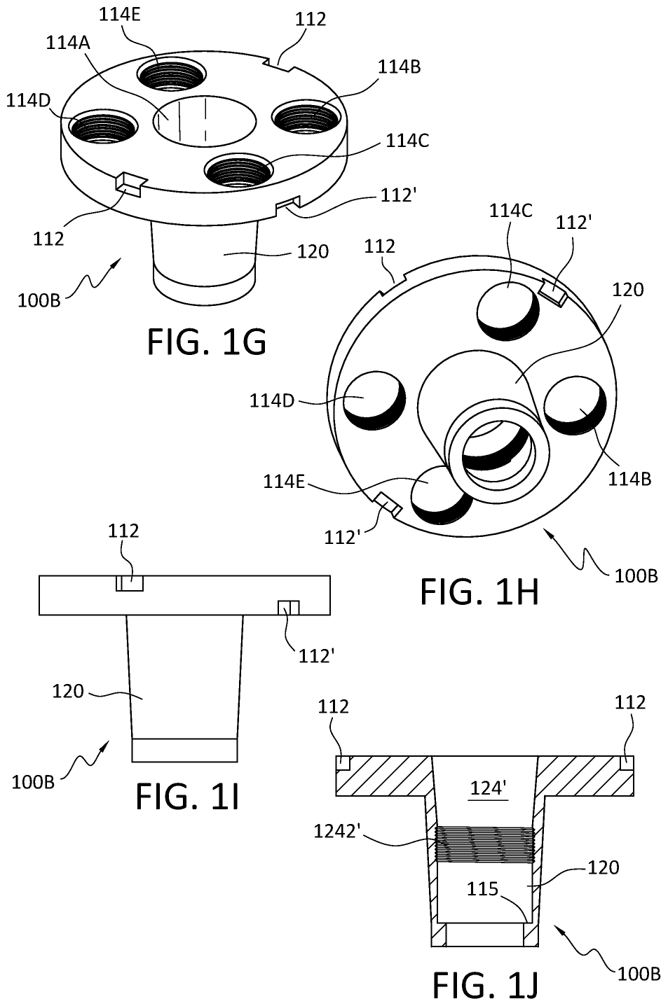

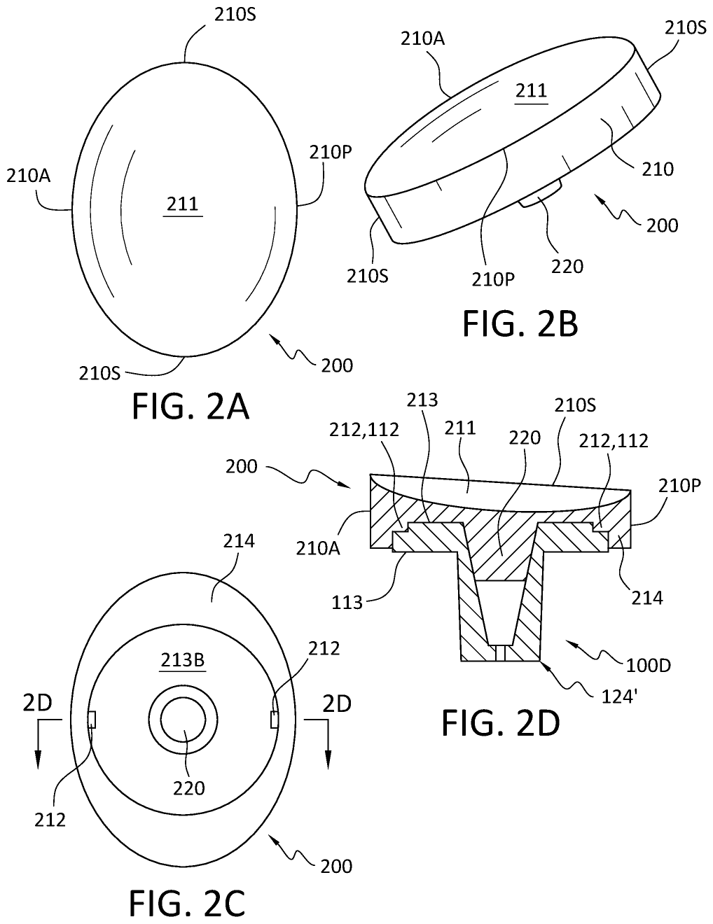

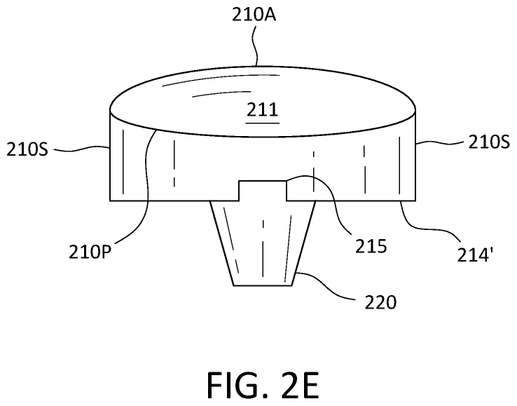

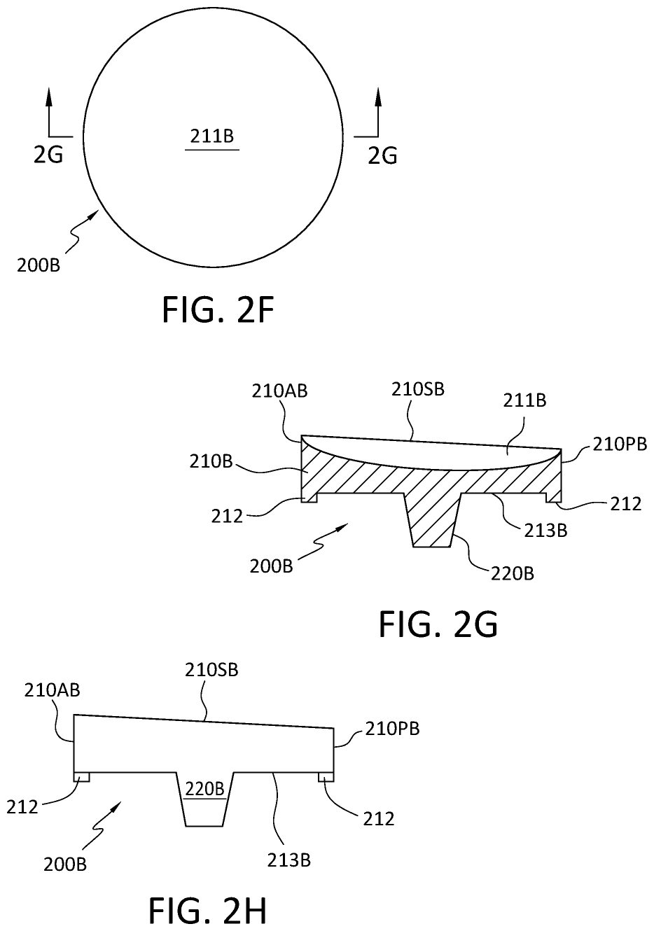

The invention in at least one embodiment includes a glenoid modular component for use with baseplates disclosed in U.S. Pat. No. 10,583,012 issued on Mar. 10, 2020, and PCT Application No. PCT/US21/20492 filed on Mar. 2, 2021 (published as WO 2021/178418 A1 on Sep. 10, 2021), which are hereby both incorporated by reference for their teachings regarding the baseplate for use on the glenoid side for any TSR. FIGS. 1A-1J illustrate examples of some of those baseplates. In at least one embodiment, the modular glenoid component as illustrated in FIGS. 2A-2H includes a base having a concave top surface and a plug extends from a surface opposite of the concave surface to be inserted into the baseplate. In at least one embodiment, the modular glenoid component includes an outer peripheral flange that overlaps with the side of the baseplate that in at least one embodiment include a pair of attachment points extending in from opposed external peripheral sides to engage notches in the baseplate.

The baseplate has a mounting surface for engagement of a modular glenoid component for a ta-TSR or a modular glenosphere component for a r-TSR. The mounting surface refers to the substantially planar surface of the baseplate opposite the glenoid (or humerus). The modular glenoid component is an example of a modular component. The attachment in at least one embodiment between the modular component and the baseplate is through, for example, a Morse taper, which may be a dual threaded Morse taper that is axially located with reference to the baseplate. In an alternative embodiment with or without the Morse taper, a torque limiting fastener, such as a screw or a bolt, is used to further secure the modular glenoid component to the baseplate by engaging the baseplate and/or the central attachment mechanism anchored in the patient's glenoid. In a further embodiment, the attachment is facilitated with a threaded connection where the modular component is screwed into the baseplate. In at least one embodiment, the baseplate and the modular-glenoid component are adapted for use on the humerus, for example in a r-TSR.

FIGS. 1A and 1B illustrate a baseplate 100. The illustrated baseplate 100 includes a base 110 and a stem 120 extending from the base 110. Although the base 110 is illustrated as being circular, the base 110 may be elliptical, oval, or other suitable shapes, in such an embodiment, the modular component (or at least the optional flange) may be shaped to match. Examples of the thickness of the base 110 include between 5 mm and 15 mm (with or without the end points), 5 mm, 7 mm, 10 mm, 12.5 mm, and 15 mm. In at least one embodiment, the baseplate 100 is made at least in part from an ingrowth trabecular metal over a metal core of, for example, steel, titanium or a combination of the two. The ingrowth trabecular metal facilitates bone ingrowth into the baseplate 100, for example to increase the strength of the connection between the bone and the baseplate 100 and the respective interface shear strength over time.

Although the stem 120 and 120′ in FIGS. 1B and 1C, respectively, are illustrated as a cylinder with a slight taper, in at least one embodiment, the stem 120 has tapered sides to match the modular component plugs. In a further embodiment, the stem 120 is substantially cylindrical.

The base 110 includes a mounting surface 111 on a side opposite of the side 113 from which the stem 120 extends. In at least one embodiment, the mounting surface 111 is substantially planar. The plane defined by the mounting surface 111 is approximately parallel to the glenoid resection plane after implantation. In an alternative embodiment, the plane defined by the mounting surface 111 is at an angle to the glenoid resection plane after implantation as illustrated, for example, in FIGS. 1D and 1E. The mounting surface 111 in at least one embodiment will be a sufficient height above the glenoid resection plane to allow access to notches 112, 112 or attachment points 112′, 112″.

The illustrated base 110 of FIGS. 1A-1C includes a pair of opposed leverage notches 112, 112 extending down from outer circumferential sides of the mounting surface 111, for example on the anterior and posterior central exterior edge (or side) of the base 110, which in at least one embodiment provides better access to the notches for removal of the modular component although the notches may be rotated 90 degrees from the anterior and posterior sides of the base 110. The notches 112, 112 are configured to be accessible from the mounting surface 111. In at least one embodiment, the notches 112, 112 provide (or are configured to have) a leverage point to facilitate separation of the mounted modular component from the baseplate 100 when the baseplate 100 is implanted. The notches 112, 112 have sufficient width and depth to receive an instrument in which to pry the mounted modular component from the baseplate 100. Although two notches 112, 112 are illustrated, additional notches could be added to the base 110. Ina further embodiment, each of the notches 112, 112 receive a protrusion 212, 212 (see, e.g., FIGS. 2C, 2D, 2F and 2G) depending from a base of the modular component, for example to increase the strength of the connection between the base 110 and the modular component 200, 300. In at least one embodiment when additional notches are present, the modular component may be rotated relative to the base to engage the two notches that provide a preferred orientation for engagement of the modular humeral component by the modular component. In a further embodiment, a space will be defined by the notch 112 and the protrusion 212 to receive an instrument to pry the mounted modular component 200, 300 from the baseplate 100. In at least one embodiment, the leverage notches 112, 112 are omitted, while in another embodiment the leverage notches are the attachment points for fingers of the instrument.

The illustrated base 110 includes a pair of attachment points, which are illustrated as notches 112′, 112′ in FIGS. 1C and 1F and slot 112″ in FIGS. 1D and 1E. In FIGS. 1C-1F, the attachment points 112′/112″ are aligned with the notches 112, 112. In at least one other embodiment illustrated in FIGS. 1G-1J, the attachment points 112′/112″ are position at 7 o'clock and 1 o'clock when the notches 112, 112 are at the anterior and posterior walls (i.e., 3 o'clock and 9 o'clock). The notches extend up from outer circumferential sides of the bottom surface 113 from which the stem 120 extends. The notches 112′ and the slots 112″ are present on the outer circumferential sides of the base 110. Both types of attachment points are accessible from the exterior of the baseplate 100. The attachment points have sufficient width and depth to engage with an implanting/extracting instrument. In at least one further embodiment, the attachment points are configured to match the shape of the instrument used for implanting and/or extracting the baseplate 100 including the cross-section and/or depth of a finger of the instrument. The instrument fingers are inserted into the attachment points 112/112′/112″ to assist with the implantation of the baseplate 100 onto the glenoid and, if necessary, is used to extract the baseplate 100 from the glenoid. Although two attachment points are discussed, additional attachment points could be added to the base 110. FIGS. 1D and 1E illustrate examples of how both a notch 112′ and a slot 112″ may be present to provide attachment points when a wedge is present.

In at least one embodiment where leverage notches are present, the leverage notches and the attachment points are aligned with each other as illustrated, for example, in FIGS. 1C-1F, while in an alternative embodiment the leverage notches and the attachment points are offset from each other as illustrated in FIGS. 1G-1J.

The illustrated base 110 includes five mounting holes 114A-114E with an axially centered hole 114A and four evenly spaced perimeter holes 114B-114E around the mounting surface 111. The holes 114A-114E are illustrated as having a shoulder 115 on which a screwhead, which screw is an example of an attachment mechanism 130, 130A, will make contact after insertion into the baseplate 100. Although five holes are illustrated, the number of holes could be reduced, including omission of the perimeter holes, or increased. In at least one embodiment as illustrated, for example, in FIG. 2D, the central hole 114A defines a chamber 124 for receiving a modular component plug 220. In at least one embodiment, the holes 114B-114E are offset from the notches 112, 112 as illustrated, for example, in FIG. 1A. For example, hole 114B might be at approximately 1 o'clock or 2 o'clock while hole 114E might be at approximately 10 o'clock or 11 o'clock if the notches 112, 112 are at 3 o'clock and 9 o'clock, respectively. FIG. 1G illustrates an alternative embodiment where the openings 114C and 114E are not offset with notches 112, 112, 112′, 112′.

In at least one embodiment, one or more variable angle locking screws 130, 130A are used to attach the baseplate 100 to the patient's glenoid bone. Examples of screw diameters include 4.5 mm to 5.0 mm and in a further example including the end points of that range, and more particularly 5.0 mm. Although there are five holes illustrated, during a particular procedure, all five holes may not be utilized. In at least one embodiment, the flexibility in which holes 114A-114E to use and the variable angle locking screws 130, 130A provides flexibility to the orthopedic surgeon in securing the baseplate 100 to the patient's glenoid bone. Examples of locking screw angles includes between 20 degrees and 30 degrees (with or without the end points) or perpendicular to the base 110. FIG. 1B illustrates a screw 130A that includes a receiving cavity 134 for insertion of a torque limiting fastener inserted through the modular component.

In at least one embodiment, the central axial opening 114A passes from the base 110 into and through the stem 120 to allow for the top of the locking screw 130A to be deeper into the baseplate 100 and to provide the chamber 124′ for receiving a plug, e.g., the Morse taper, of the modular component being mounted onto the baseplate 100. FIG. 1C illustrates the chamber 124′ having receiving screw threads for engaging a torque limiting fastener 330′. As referenced above, the torque limiting fastener may engage locking screw 130A instead or in addition to other areas of the chamber 124′. FIG. 1C illustrates the modular component as the glenosphere component 300 with a glenosphere 316 and a passageway 324. In at least one embodiment, the modular components illustrated in FIGS. 2A-2H include a passageway like passageway 324. In a further alternative embodiment, the modular component may include protrusions depending down from the bottom surface configured to have an interference fit with one or more of openings 114B-114E similar to the plug be received in chamber 124/124′.

FIGS. 1D and 1E illustrate a pair of alternative baseplates 100A, 100B that have a partial wedge 116A and a full wedge 116B, respectively. One of ordinary skill in the art should appreciate that the presence of a wedge is potentially advantageous for addressing bone deformities of the glenoid while providing a secure attachment of the baseplate 100 to the patient's glenoid bone. Alternatively, the baseplate 100 can have a 10 degree or 25 degree back as oppose to the neutral back illustrated in FIG. 1B. In embodiments with a wedge, the attachment points may be a combination of a slot 112″ on one side and a notch 112′ on the opposing side to the wedge may be added to the baseplates 100A, 100B. Also illustrated in FIGS. 1D and 1E are optional notches 112, 112.

FIGS. 1G-1I illustrate an alternative baseplate 1000 with notches 112, 122 at approximately 3 o'clock and 9 o'clock, respectively, and attachment points 112′, 112′ at approximately 5 o'clock and 11 o'clock, respectively. FIG. 1J illustrates the presence of a threaded section 1242′ present in chamber 124′ of the plug 120. Otherwise, FIGS. 1G-1I mirror the previously discussed FIGS. 1A-1C and 1F.

FIGS. 2A-2H illustrate multiple examples of modular glenoid components 200, 200B each having a concave (or articulating) surface 211, 211B on a base 210, 210B for receiving a humeral head (e.g., a prosthetic ball, spherical object, or another convex shaped interface) and a plug 220, 220B extending from a surface 213, 213B opposed to the concave surface 211, 211B on the base 210, 210B. The concave surface 211, 211B includes an angle such that the anterior wall 210A, 210AB is higher than the posterior wall 210P, 210PB with the side walls 210S, 210SB having a general angle to them to adjust the overall wall height between the anterior and posterior sides of the modular glenoid component. In at least one embodiment, the angle is measured by a phantom line taken from the anterior wall top to the posterior wall top relative to a horizontal plane passing through the base that is parallel to the bottom surface(s) of the base, flange, and/or plug depending on the particular embodiment. FIG. 2H provides an illustration of the general angle of the exterior of the side wall that in at least one embodiment would be parallel to the phantom line. The configuration for the anterior and posterior walls 210A, 210P (or 210AB, 210PB) protects against anterior instability to prevent humeral head sublocation anteriorly. In at least one embodiment, the anterior wall 210A, 210AB is approximately 3 mm higher than the posterior wall 210P, 210PB, or alternatively the height differential is in a range of approximately 2.5 mm to approximately 3.2 mm.

The plug 220, 220B is configured to be inserted and frictionally engage the chamber 124 (or 124′ in FIG. 2D) in the baseplate 100 (or baseplate 100D in FIG. 2D). In at least one embodiment, the plug is a Morse taper central plug.

One of ordinary skill in the art should appreciate based on this disclosure, the concave surface can take a variety of shapes without departing from the modularity of the modular glenoid component 200, 200B. An example of a different shape is a variating surface with a non-uniform level of curvature over the concave surface. In at least one embodiment, the maximum depth of the concave surface measured from a line drawn between the anterior and posterior sides is in a range of 1.5 mm to 3 mm, or in a further embodiment approximately 2 mm. In at least one embodiment, the thickness of the base 210, 210B at center of the concave surface is between 3 mm and 4 mm, which would have a thickness of the base 210, 210B (without the presence of any flange or protrusion) at the side walls 210S, 210SB would be between 6 mm and 7 mm resulting in the glenoid bone being approximately 7 mm to approximately 8 mm from the top of the side walls 210S, 210SB.

Although the modular glenoid component 200 is illustrated as being elliptical with the anterior and posterior sides being perpendicular to the minor axis in FIGS. 2A-2D, the concave surface 211B may be round, oval or other similar shapes when viewed from the top as viewed in FIG. 2A or FIG. 2E. FIGS. 2F-2H illustrate the modular glenoid component 200B with the base 210B and the concave surface 211B being round.

FIGS. 2C, 2D, 2G, and 2H illustrate the modular glenoid component 200, 200B including a pair of protrusions 212, 212 depending from the bottom surface 213, 213B proximate to the anterior and posterior sides 210A/210AB, 210P/210PB. In at least one embodiment, the protrusions 212, 212 are aligned with the notches 112, 112 of the baseplate 100D after the modular glenoid component 200, 200B is implanted, but in an alternative embodiment the protrusions 212, 212 and notches 112, 112 are rotated 180 degrees. Protrusions 212, 212 are shaped to be inserted into and engage at least a portion of a corresponding notch 112. In at least one further embodiment, the protrusion 212 and the notch 112 have an interference fit. As discussed above, the notch 112 and the protrusion 212 may define a space for receiving an instrument, or alternatively the protrusion 212 fully fills the space of the notch 112. In an alternative embodiment, the protrusions 212, 212 are omitted. In a further alternative embodiment, the protrusions extend radially out from the anterior and posterior sides. In another alternative embodiment, the protrusions are combined together to both radially extend out and extend down from the base. In at least one embodiment where the protrusions 212 are present and the leverage notch is also the attachment point, fingers of the instrument will reach below at least one protrusion to engage the modular component. In a further embodiment, the finger will have a step in it such to engage the space between protrusion 212 and the notch 112 with a step to then engage the protrusion 212 to provide additional leverage. In a further alternative embodiment with a flange for the modular component, the protrusion 212 extends radially out from the peripheral side of the base and/or a flange 214.

FIGS. 2C and 2D illustrate the modular glenoid component having an optional flange 214 extending down from the bottom surface 213 of the base 210 and a pair of protrusions 212 extending down from the base 210 and in from an interior side of the flange 214. In at least one embodiment, the flange 214 is the remaining body of the glenoid component when the central circle equal to the baseplate is cut-away (or otherwise removed) to allow for the glenoid component to interlock and seat onto the baseplate. In FIGS. 2D and 2E, the flange 214, 214′ is configured to fit over and down at least a portion of the peripheral external sides of the baseplate 100D. In this embodiment, the base 210 has a wider diameter than the baseplate 100D. In at least one embodiment as illustrated in FIG. 2E, the flange 214′ includes at least two slots 215 (one is illustrated in FIG. 2E) extending up from the bottom of the flange 214′ toward the base 210, which in at least one embodiment are configured to align with the attachment points of the baseplate. FIGS. 2G and 2H illustrate how the protrusions 212, 212 would look without the presence of the optional flange.

Although the concave surface is elliptical, the cavity defined by the flange 214 is configured to fit over the base plate 100D as illustrated in FIG. 2D. FIG. 2D also illustrates the engagement of the protrusions 212 into the notches 112. Although not illustrated, the plug 220 could be cylindrical shape to provide additional interference fit with the chamber 124′ of the baseplate 100D.

Although in FIG. 2D the bottom of the flange 214 is shown as being spaced from the bottom outer edge of the baseplate 100D, these surfaces may be flushed with each other. In an embodiment where the space is present, the bottom of the flange 214 is between 1 mm and 2 mm from the bottom outer edge of the baseplate 100D.

In at least one embodiment, the top rim of the modular component 200, 200B includes a rounded or substantially flat surface from the concave top surface to the exterior of the side wall.

In at least one embodiment, the modular glenoid component is manufactured from Cobalt-Chromium (Co—Cr) to improve the life expectancy for the implant.

In a further embodiment to any of the embodiments discussed above having protrusions that have an interference fit with notches, the baseplate may omit a receiving cavity and instead rely on the interference fit between the protrusions and the notches to secure the attached modular component when the system is implanted in a patient. In such an embodiment, the plug would be omitted from the modular glenoid component.

Although particular materials have been identified for particular components and structural elements, one of ordinary skill in the art will appreciate that other materials may be substituted without departing from the scope of the invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention.

As used above “substantially,” “generally,” “approximately,” and other words of degree are relative modifiers intended to indicate permissible variation from the characteristic so modified particularly when relating to manufacturing and production tolerances and orthopedic surgery results. It is not intended to be limited to the absolute value or characteristic which it modifies but rather possessing more of the physical or functional characteristic than its opposite, and preferably, approaching or approximating such a physical or functional characteristic. The use of ranges covers both including the end points (absolute or approximate) and not including the end points.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

Although one level of multiple dependencies is present in the claims attached to this disclosure, it should be understood that none conflicting claim recitation of dependent claims may be combined, for example, in the manner of the priority application.

1. A modular glenoid component for use in a modular shoulder prosthesis system having a baseplate with notches and/or attachment points, the modular glenoid component comprising:
a base having a concave top surface angled from an anterior side to a posterior side such that said anterior side is higher than said posterior side, and
a plug extends from a surface opposite of said concave top surface, said plug is configured for insertion into the baseplate.

2. The modular glenoid component according to claim 1, further comprising a peripheral flange that extends down to fit over outer circumferential sides of the baseplate when installed providing an interference fit between the modular glenoid component and the baseplate.

3. A modular glenoid component for use in a modular shoulder prosthesis system having a baseplate with notches and/or attachment points, the modular glenoid component comprising:
a base having a concave top surface angled from an anterior side to a posterior side such that said anterior side is higher than said posterior side, and
a peripheral flange that extends down to fit over outer circumferential side of the baseplate when installed providing an interference fit between the modular glenoid component and the baseplate.

4. The modular glenoid component according to claim 3, wherein said flange includes a pair of opposed slots extending up from a bottom of said flange, said slots configured to align with the attachment points of the baseplate and to provide a leverage point on which to apply leverage for removal of said modular component after installation onto the baseplate.

5. The modular glenoid component according to claim 3, wherein said concave top surface has a maximum depth measured from a phantom line from said anterior side to said posterior side of between 1.5 mm and 3 mm.

6. The modular glenoid component according to claim 3, wherein said concave top surface is a variating surface with a non-uniform level of curvature over said concave top surface.

7. The modular glenoid component according to claim 3, further comprising a pair of protrusions extending from opposing sides of said base, said protrusions configured to align with the notches extending down from a mounting surface of the baseplate when said modular glenoid component is attached to the baseplate and/or configured to engage with an interference fit the notches when said modular glenoid component is attached to the baseplate.

8. (canceled)

9. (canceled)

10. The modular glenoid component according to claim 1, wherein said concave top surface has a maximum depth measured from a phantom line from said anterior side to said posterior side of between 1.5 mm and 3 mm.

11. The modular glenoid component according to claim 10,
wherein said concave top surface is a variating surface with a non-uniform level of curvature over said concave top surface.

12. (canceled)

13. (canceled)

14. The modular glenoid component according to claim 3, wherein said glenoid component is manufactured from Cobalt-Chromium.

15. The modular glenoid component according to claim 3, wherein said concave top surface is a variating surface with a non-uniform level of curvature over said concave top surface.

16. (canceled)

17. (canceled)

18. The modular glenoid component according to claim 1, wherein said glenoid component is manufactured from Cobalt-Chromium.

19. A modular shoulder prosthesis system comprising:
a baseplate having
a base with at least one attachment hole passing therethrough and
a pair of attachment points on opposed outer circumferential sides and below a mounting surface of said base, said attachment points extend at least in from said outer circumferential sides of said base, and/or
a pair of leverage notches on opposed outer circumferential sides of said base, said leverage notches extend down from a mounting surface of said baseplate and in from said outer circumferential sides of said base, and
a central stem extending from a bottom surface of said base and axially centered with an optional one of said at least one attachment hole;
a modular component according to claim 1 configured to be removably attached to said base of said baseplate, and
wherein said baseplate is capable of attachment to different modular components to facilitate both traditional anatomic total shoulder replacement and reverse total shoulder replacement with a change in the modular component; and
said attachment points are configured to provide leverage points to facilitate extraction of said baseplate from a patient; and
said modular component is manufactured from Cobalt-Chromium.

20. The system according to claim 19, wherein said leverage notches are on the anterior and posterior sides of said base and said attachment points are laterally offset from said leverage notches.

21. (canceled)

22. (canceled)

23. (canceled)

24. (canceled)

25. (canceled)

26. (canceled)

27. A modular glenoid component for use with a baseplate of a modular prosthesis system, the modular glenoid component comprising:
means for engaging the baseplate after the baseplate has been implanted in a patient;
means for assisting in a detachment of the modular glenoid component from the baseplate; and
the modular glenoid component is an unibody structure including Cobalt-Chromium.

28. The modular glenoid component according to claim 27, wherein said unibody structure includes a base having a concave top surface angled from an anterior side to a posterior side such that said anterior side is higher than said posterior side.

29. The modular glenoid component according to claim 28, wherein
said concave top surface has a maximum depth measured from a phantom line from said anterior side to said posterior side of between 2 mm and 3 mm; and
said concave top surface is a variating surface with a non-uniform level of curvature over said concave top surface.

30. The modular glenoid component according to claim 1, wherein
said concave top surface has a maximum depth measured from a phantom line from said anterior side to said posterior side of between 2 mm and 3 mm; and
said concave top surface is a variating surface with a non-uniform level of curvature over said concave top surface.

31. The modular glenoid component according to claim 1, wherein the top surface is elliptical shaped with a minor axis between said anterior side and said posterior side.

32. The modular glenoid component according to claim 3, wherein the top surface is elliptical shaped with a minor axis between said anterior side and said posterior side.
